(19) United States
(12) Patent Application Publication
(10) Pub. No.: US 2026/0133112 A1
(43) Pub. Date: May 14, 2026

(54) CONTROL CELL POPULATIONS FOR DETECTION OF PAROXYSMAL NOCTURNAL HEMOGLOBINURIA

(71) Applicant: BioLegend, Inc., San Diego, CA (US)

(72) Inventors: Susannah Helene Kassmer-Rodriguez, San Diego, CA (US); Anagha Ashok Divekar, San Diego, CA (US); David Pierre Michel Pillon, San Diego, CA (US)

(21) Appl. No.: 19/385,304

(22) Filed: Nov. 11, 2025

Related U.S. Application Data
(60) Provisional application No. 63/719,211, filed on Nov. 12, 2024.

(51) Int. Cl. G01N 15/14; G01N 15/10; G01N 33/542; G01N 33/569

(52) U.S. Cl. CPC G01N 15/14; G01N 33/542; G01N 33/56972; G01N 2015/1006; G01N 2015/1488; G01N 2333/70535; G01N 2333/70589; G01N 2333/70596

(57) ABSTRACT

Provided are systems and methods that may be employed to detect the presence or absence of paroxysmal nocturnal hemoglobinuria (PNH) in a subject that includes a leukocyte including GPI-anchors the leukocyte incubated with one or more blocking agents each independently specific to one or more GPI-anchored proteins, the leukocyte further incubated with a pro-aerolysin or derivative thereof, and optionally a second leukocyte including GPI-anchors not incubated with one or more of the blocking agents. The systems may be used as a positive control in assays detecting the presence or absence of PNH.

### CROSS REFERENCE TO RELATED APPLICATIONS

This application depends from and claims priority to U.S. Provisional Application No. 63/719,211 filed Nov. 12, 2024, the entire contents of which are incorporated herein by reference.

### FIELD

The present description relates to systems, methods, and control cell populations for studies involving detection of the presence or absence of a disease or condition.

### BACKGROUND

Paroxysmal nocturnal hemoglobinuria (PNH) is an acquired clonal blood disorder characterized by an inability to properly produce glycosylphosphatidylinositol (GPI)-anchors that bind to proteins such as the complement-defense structures CD55 and CD59 on red blood cells or white blood cells. PNH results from mutation of the phosphatidylinositol glycan complementation Class A gene that encodes an enzyme involved in the first stage of glycosylphosphatidylinositol biosynthesis.

Subjects with PNH suffer intravascular hemolysis, thrombosis and bone marrow failure. There are few treatments for PNH, but the use of monoclonal antibody therapy targeting the terminal complement protein C5 has recently been approved. This antibody successfully reduces hemolysis, transfusion requirements and thrombosis. Thus, a key to properly treating a population with PNH is successful and early identification of the presence of the disease and the ability to properly monitor any treatment.

Flow cytometry is the gold standard method currently used for the diagnosis of PNH. These studies detect the absence of GPI-linked protein expression on granulocyte and monocyte surfaces. Since this disease is extremely rare, however, clinical laboratories do not have easy access to control samples from diseased patients and have to rely on other ways to validate the diagnostic flow cytometry assay.

Thus, there is a continuing need for improved compositions and methods for improving the detection of PNH and any disease or condition that can benefit from the materials and processes as provided herein.

### SUMMARY

The following summary of the disclosure is provided to facilitate an understanding of some of the innovative features unique to the disclosure and is not intended to be a full description. A full appreciation of the various aspects of the disclosure can be gained by taking the entire specification, claims, drawings, and abstract as a whole.

Provided herein according to some aspects are systems configured for use in detection of the presence or absence of paroxysmal nocturnal hemoglobinuria including: a leukocyte comprising GPI-anchors said leukocyte incubated with one or more blocking agents each independently specific to one or more GPI-anchored proteins; the leukocyte further incubated with a pro-aerolysin or derivative thereof; and optionally a second leukocyte comprising GPI-anchors not incubated with one or more of the antibodies. In some aspects, one or more of the blocking agents is specific for CD157, CD64, CD15, CD45, CD24, or CD14, optionally CD157, CD24, or CD14. The leukocytes may include a plurality of monocytes, granulocytes, or a combination thereof, wherein in some aspects, the leukocytes are monocytes. Optionally, the leukocytes may be human leukocytes. Optionally, the leukocytes are previously lyophilized leukocytes.

Also provided are systems configured for use in detection of the presence or absence of paroxysmal nocturnal hemoglobinuria including: a leukocyte comprising GPI-anchors; one or more antibodies each independently specific to one or more GPI-anchored proteins; and a pro-aerolysin or derivative thereof. In some aspects, one or more of the blocking agents is specific for CD157, CD64, CD15, CD45, CD24, or CD14, optionally CD157, CD24, or CD14. The leukocytes may include a plurality of monocytes, granulocytes, or a combination thereof, wherein in some aspects, the leukocytes are monocytes. Optionally, the leukocytes may be human leukocytes. Optionally, the leukocytes are previously lyophilized leukocytes. In some aspects, the system further includes one or more buffers, a lysing agent, or a combination thereof.

Also provided are processes of detecting the presence or absence of paroxysmal nocturnal hemoglobinuria (PNH) in a subject including: obtaining a first sample of whole blood from the subject, the sample optionally lysed, fixed, and washed, the first sample including one or more leukocytes; contacting the first sample with a blocking solution including one or more blocking agents (optionally antibodies) each independently specific to one or more GPI-anchored proteins and a pro-aerolysin or derivative thereof, incubating the sample wit the blocking solution for a first incubation time to form a blocked sample; combining the blocked sample with a PNH antibody cocktail; and subsequently subjecting said blocked sample to analysis by flow cytometry to detect the presence or absence of paroxysmal nocturnal hemoglobinuria in the subject. In some aspects, the process further includes obtaining a second sample of whole blood from the subject, the second sample optionally a portion of the first sample; combining the second sample with the PNH antibody cocktail; and intermixing the second sample with the blocked sample prior to subjecting the blocked sample to analysis by flow cytometry. In some aspects, one or more of the blocking antibodies is specific for CD157, CD64, CD15, CD45, CD24, or CD14, optionally CD157, CD24, or CD14. Optionally, the PNH antibody cocktail includes a labeled antibody specific for CD157, CD64, CD15, CD45, CD24, CD14, or a combination of such antibodies. Optionally, the PNH antibody cocktail includes six labeled antibodies, wherein each of the six antibodies are independently specific for CD157, CD64, CD15, CD45, CD24, and CD14.

Also provided are processes of analyzing a positive control for paroxysmal nocturnal hemoglobinuria (PNH) including: obtaining a leukocyte comprising GPI-anchors, said leukocyte incubated with one or more antibodies each independently specific to one or more GPI-anchored proteins; further incubating the leukocyte with a pro-aerolysin or derivative thereof to form a blocked leukocyte; further incubating the blocked leukocyte with a PNH antibody cocktail; combining the blocked leukocyte with an unblocked leukocyte to form a leukocyte mix; and subjecting said leukocyte mix to analysis by flow cytometry. Optionally, the leukocyte or the unblocked leukocyte includes a plurality of monocytes, granulocytes, or a combination thereof. Optionally, the blocked leukocytes and/or unblocked leukocytes are human leukocytes. Optionally, the blocked leukocytes and/or unblocked leukocytes are previously lyophilized leukocytes. In some aspects, one or more of the blocking antibodies is specific for CD157, CD64, CD15, CD45, CD24, or CD14, optionally CD157, CD24, or CD14. Optionally, the PNH antibody cocktail includes a labeled antibody specific for CD157, CD64, CD15, CD45, CD24, CD14, or a combination of such antibodies. Optionally, the PNH antibody cocktail includes six labeled antibodies, wherein each of the six antibodies are independently specific for CD157, CD64, CD15, CD45, CD24, AND CD14.

Also provided are kits that may be used for detection of the presence or absence of paroxysmal nocturnal hemoglobinuria (PNH) in a subject including: a first container and a second container, the first container including an unblocked leukocyte, the unblocked leukocyte incubated with a PNH antibody cocktail, and wherein the second container includes a blocked leukocyte, the blocked leukocyte incubated with one or more antibodies to one or more GPI-anchored proteins, a pro-aerolysin or derivative thereof, and with the PNH antibody cocktail.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings.

FIG. 1 illustrates flow cytometry gating on lymphocytes (A) and individual granulocytes (B) or monocytes (C) from whole blood obtained according to one aspect as provided herein.

FIG. 2 illustrates the positive detection of blocked granulocytes gated per FIG. 1 according to one aspect as provided herein.

FIG. 3 illustrates the positive detection of blocked monocytes gated per FIG. 1 according to one aspect as provided herein.

FIG. 4 illustrates that previously lyophilized leukocytes can be successfully employed in a process for detection of PNH as provided herein according to one aspect.

FIG. 5 illustrates an exemplary process for using previously lyophilized leukocytes in a process as provided herein according to one aspect.

### DETAILED DESCRIPTION

Provided herein are cell populations that are able to successfully mimic cells of a subject that are lacking one or more proteins on the surface relative to a normal cell population and are useful for the detection of the presence or absence of a disease or condition. By selectively blocking binding of one or more diagnostic antibodies to the surface of a cell, the cells as provided herein mimic a disease phenotype and can be readily used as a control in assays for detection of the presence of a disease or condition.

The systems and processes described herein may be applicable to detect any disease or condition in which a leukocyte expresses one or more downregulated surface epitopes. Illustrative examples of a disease or condition include PNH, Alzheimer's disease, leukocyte adhesion deficiency type 1 (LAD1), autoimmune lymphoproliferative syndrome (ALPS), CD40 deficiency, or Mendelian susceptibility to mycobacterial diseases (MSMD).

Provided herein according to some aspects is a system configured for use in detection of the presence or absence of PNH in a subject. The system includes one or more leukocytes including GPI-anchors wherein the leukocytes are incubated with one or more blocking agents each independently specific to one or more GPI-anchored proteins and further wherein the one or more leukocytes is further incubated with a pro-aerolysin or derivative thereof.

A system as provided herein includes one or more leukocytes. Optionally, a system as provided herein includes a plurality of leukocytes, optionally where each leukocyte in the plurality of leukocytes share a common protein expression on the surfaces thereof such that all or substantially all of the leukocytes are of the same phenotype. GPI-anchors are found on the outer leaflet of the lipid bilayer of the one or more leukocytes facing the extracellular environment. The GPI-anchors may each individually be bound to a protein via the carboxy-terminus thereby anchoring the extracellular protein to the leukocyte plasma membrane. All known GPI anchors share a common core of ethanolamine-PO₄-6Manα1-2Manα1-6Manα1-4GlcNα1-6myo-inositol-1-PO₄-lipid where “Man” is mannose and “GlcN” is glucosamine. A Man at the distal end is attached to the protein via an ethanolamine phosphate (EtNP) bridge between its C-6 hydroxyl and the α-carboxyl group of the carboxy-terminal amino acid. Other than the common core, it is known in the art that GPI-anchors are diverse and related to the protein to which it is attached and the species from which the anchor is produced. (Komath, et al., Essentials of Glycobiology [Internet], 4th edition, 2022, The Consortium of Glycobiology Editors, La Jolla, California, Cold Spring Harbor Press, doi:10.1101/glycobiology.4e.12) For example, the PI in GPI is D-myo-inositol-1-P-3(sn-1,2-diacylglycerol) (i.e., diacyl-PI), but may instead be other inositolphospholipids, such as lysoacyl-PI, alkylacyl-PI, alkenylacyl-PI, and inositolphosphoceramide. Id. The leukocytes in the system as provided herein optionally have a complement of GPI-anchors anchored to proteins as are found in non-diseased human subjects, optionally human subjects that are free from a disease or condition that alters the GPI-anchor distribution or the presence of the proteins that are bound thereto. Optionally, the GPI anchors are as found on leukocytes from a human that is free from PNH.

A leukocyte is optionally a leukocyte that is produced by an organism or is produced so as to have a complement of GPI-anchors and associated bound proteins that mimic one or more such anchors/proteins as is found in that organism. An organism from which the leukocytes are derived is optionally a human or non-human primate. Optionally, an organism is a bovine, equine, porcine, murine, or other mammal or a non-mammal. In some aspects, an organism from which a leukocyte is derived is a human.

A leukocyte optionally is or includes one or more monocytes. Optionally, a leukocyte is or includes one or more granulocytes. Optionally, a leukocyte is a plurality of leukocytes that may be monocytes, granulocytes, or a combination thereof. In some aspects, a plurality of leukocytes are or include monocytes. The monocytes, granulocytes or combination thereof are optionally human monocytes, granulocytes or a combination thereof.

In some aspects, a leukocyte is a previously lyophilized leukocyte. The leukocytes may be lyophilized or lyophilized previously and reconstituted in a reconstitution buffer. Illustrative examples of lyophilized leukocytes are sold as VERI CELLS by Biolegend, Inc. (San Diego, CA). Optionally, the lyophilized leukocytes are VERI CELLS leukocytes sold by Biolegend, Inc. (San Diego, CA).

A leukocyte as provided herein is in some aspects bound to one or more blocking agents whereby a blocking agent is a composition that is able to selectively bind a GPI-anchored protein on the surface of the leukocyte. A blocking agent may be a protein, nucleic acid (double or single stranded DNA, RNA, or other), hormone, neurotransmitter, sugar, organic or inorganic small molecule (e.g. 1000 Da or smaller), or other material that is able to selectively bind a GPI-anchored protein. In some aspects, a blocking agent is an antibody, a fragment thereof, or a derivative thereof.

A blocking agent is any molecule that may bind a GPI-anchored protein, and optionally, selectively bind a GPI-anchored protein. An illustrative example of a blocking agent includes, but is not limited to an antibody, such as any suitable antibody known in the art including other immunologically active fragments of antibodies or single chain antibodies. A blocking agent may be a single moiety, e.g., a polypeptide or protein, or it may include two or more moieties, e.g., a pair of polypeptides such as a pair of single chain antibody domains. An antibody may be a monoclonal antibody or a polyclonal antibody. An antibody or fragment thereof may be derived from an organism such as sheep, goat, rabbit or rat. An antibody may be a monoclonal antibody or a polyclonal antibody.

A blocking agent may be an antibody fragment such as single chain antibodies (scFv), Fab and scFv antibodies, single domain antibodies (VHH), or chimeric antibodies. The blocking agent may be derived from a naturally occurring protein or polypeptide; it may be designed de novo, or it may be selected from a library. Optionally, a blocking agent may be derived from an antibody, a single chain antibody (scFv), a single domain antibody (VHH), a lipocalin, a single chain MHC molecule, an Anticalin™ (Pieris), an Affibody™, a nanobody (Ablynx) or a Trinectin™ (Phylos).

In some aspects, a blocking agent is a fragment of an antibody, optionally an antigen binding fragment or a variable region. Examples of antibody fragments include Fab, Fab′, F(ab′)₂ and Fv fragments. Additional blocking agent molecules include diabodies, linear antibodies, single-chain antibody molecules, and multispecific antibodies formed from antibody fragments.

A blocking agent may be specific for one or more proteins on the surface of the leukocyte. “Specific” is defined as not appreciably selectively binding a protein other than the target protein. Optionally, specific means the blocking agent has a K_{D} for any protein other than the target protein that is 1000-fold greater or higher the K_{D} for the target protein.

A blocking agent may be specific for any particular disease or condition. For example, when detecting PNH a blocking agent may be specific for CD157, CD24, or CD14. For Alzheimer's Disease, a blocking agent may be specific for CD11b, CD11c, CD18, CD33, CD36, CD163 or any combination thereof. For LAD1, the blocking agent may be specific for CD18. For ALPS, the blocking agent may be specific for CD4 and/or CD8. For CD40 deficiency, the blocking agent may be specific for CD40. For MSMD, the blocking agent may be specific for IL-12Rβ1 or IFNγR1 or a combination thereof.

In some aspects, a blocking agent may be specific for a GPI-anchored protein selected from the group of CD157, CD64, CD15, CD45, CD24, or CD14. Optionally, a blocking agent may be specific for a GPI-anchored protein selected from the group of CD157, CD24, or CD14. In some aspects, the leukocyte(s) is incubated with a blocking agent specific to CD157, a blocking agent specific to CD24, and a blocking agent specific to CD14.

In some examples, multiple clones of each of the above antibodies may be used. Optionally, the clones used are W21007F for anti-CD157, 2D1 for anti-CD45, S18012C for anti-CD64, HI98 for anti-CD15, ML5 for anti-CD24, and S18004B for anti-CD14. Optionally, clone M5E2 of anti-CD14 is not used. Optionally, clone W20001B anti-CD24 is not used. Optionally, clone 63D3 anti-CD14 may be used.

In some aspects, an antibody is an anti-CD157 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD45 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD64 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD15 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD24 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD14 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD18 antibody. Such antibodies are commercially available such as from Biolegend, Inc. (San Diego, CA) or Thermo Fisher Scientific Inc. (Waltham, MA).

In some aspects, an antibody is an anti-CD4 antibody. Such antibodies are commercially available such as from R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD8 antibody. Such antibodies are commercially available such as from Miltenyi Biotec, Inc. (Auburn, CA), R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-CD40 antibody. Such antibodies are commercially available such as from R&D Systems, Inc. (Minneapolis, MN), Thermo Fisher Scientific Inc. (Waltham, MA), or Santa Cruz Biotechnology, Inc. (Santa Cruz, CA).

In some aspects, an antibody is an anti-IL-12Rβ1 antibody. Such antibodies are commercially available such as from R&D Systems, Inc. (Minneapolis, MN), Biolegend, Inc. (San Diego, CA), Thermo Fisher Scientific Inc. (Waltham, MA), or Santa Cruz Biotechnology, Inc. (Santa Cruz, CA).

In some aspects, an antibody is an anti-IFNγR1 antibody. Such antibodies are commercially available such as from R&D Systems, Inc. (Minneapolis, MN), Biolegend, Inc. (San Diego, CA), or Thermo Fisher Scientific Inc. (Waltham, MA).

Optionally, a leukocyte is a monocyte and a blocking agent is an anti-CD157 antibody and a CD14 antibody. Alternatively, a leukocyte is a granulocyte and a blocking agent is an anti-CD157 antibody and an anti-CD24 antibody. Optionally, a blocking agent must be or include an agent specific for CD157, optionally an antibody or fragment thereof specific for CD157. Optionally, a blocking agent must be or include an agent specific for CD157 and an agent specific for CD14. Optionally, a blocking agent must be or include an agent specific for CD157 and an agent specific for CD24.

An antibody, or other blocking agent, may be linked to one or more dyes so as to form a labeled antibody or labeled blocking agent. A dye as used herein may be a fluorophore. A fluorophore may be a rhodamine dye, a fluorescein dye, a coumarin dye, an indole dye, or any combination thereof. Optionally, a dye may be or include fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, 2-[3-[3-[6-[(2,5-dioxo-1-pyrrolidinyl)oxy]-6-oxohexyl]-2(3H)-benzoxazolylidene]-1-propen-1-yl]-3-ethyl- benzoxazolium, monoiodide (Cy2), Sulfo-Cyanine3 (Cy3), Cyanine 3.5 (Cy3.5), Sulfo-Cyanine5 (Cy5), Cyanine 5.5 (Cy5.5), Sulfo-Cyanine7 (Cy7), Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, 7-amino-3-[2-(2,5-dioxopyrrolidin-1-yl)oxy-2-oxoethyl]-4-methyl-2-oxochromene-6-sulfonic acid (Alexa Fluor® 350), N,N-Diethylethanaminium-[9-{6-[(2,5-dioxo-1-pyrrolidinyl)oxy]-6-oxohexyl}-8,8-dimethyl-2-oxo-4- (trifluormethyl)-8,9-dihydro-2H-pyrano[3,2-g]chinolin-6-yl]methansulfonate (Alexa Fluor® 430), dilithium; 3-(3-amino-6-imino-5-sulfo-4-sulfonatoxanthen-9-yl)-4-carboxybenzoate (Alexa Fluor® 488), dihydrogen 5-(4-{[(2,5-dioxo-1-pyrrolidinyl)oxy]carbonyl}phenyl)-2,3,3,7,7,8-hexamethyl-2,3,7,8-tetrahydro-1H-pyrrolo[3′,2′: 6,7]chromeno[3,2-f]indole-10,12-disulfonate (Alexa Fluor® 532), sodium 6-(2-carboxy-3,4,6-trichloro-5-{[2-({6-[(2,5-dioxopyrrolidin-1-yl)oxy]-6-oxohexyl}amino)-2-oxoethyl]thio}phenyl)-2,2,4,8,10,10-hexamethyl-3,4,5a,8,9,10,11,12a-octahydro-2H-pyrano[3,2-g:5,6-g′]diquinolin-1-ium-12,14-disulfonate (Alexa Fluor® 546), 4-(2,5-dioxopyrrolidin-1-yl)oxycarbonyl-2-[7,7,19,19-tetramethyl-9,17-bis(sulfomethyl)-2-oxa-6,20- diazapentacyclo[12.8.0.03,12.05,10.016,21]docosa-1(14),3,5,8,10,12,15,17,21-nonaen-13-yl]benzoic acid (Alexa Fluor® 568), [13-[2-carboxy-4-(2,5-dioxopyrrolidin-1- yl)oxycarbonylphenyl]-6,7,7,19,19,20-hexamethyl-17-(sulfomethyl)-2-oxa-20-aza-6-azoniapentacyclo[12.8.0.03,12.05,10.016,21]docosa-1(14),3,5,8,10,12,15,17,21-nonaen-9-yl]methanesulfonate (Alexa Fluor® 594), Alexa Fluor® 633 found at CHEBI:137393, 3-[(2Z)-2-[(2E,4E)-5-[3,3-dimethyl-5-sulfo-1-(3-sulfopropyl)indol-1-ium-2-yl]penta-2,4-dienylidene]-3-[5-(2,5-dioxopyrrolidin-1-yl)oxy-5-oxopentyl]-3-methyl-5-sulfoindol-1-yl]propane-1-sulfonate (Alexa Fluor® 647), Alexa Fluor 660, Alexa Fluor 680, 7-amino-4-methylcoumarin-3-acetic acid, (3-{2-[(3,5-Dimethyl-1H-pyrrol-2-yl-kappaN)methylene]-2H-pyrrol-5-yl-kappaN}propanoato)(difluoro)boron (BODIPY® FL), BODIPY FL-Br2, CAS No. 216961-93-2 (BODIPY® 530/550), 12-(2,2-difluoro-12-thiophen-2-yl-3-aza-1-azonia-2-boranuidatricyclo[7.3.0.03,7]dodeca-1(12),4,6,8,10-pentaen-4-yl)dodecanoate; hydron (BODIPY® 558/568), CAS#:150173-90-3 (BODIPY® 564/570), CAS#:150173-78-7 (BODIPY® 576/589), CAS Number: 217075-36-0 (BODIPY® 581/591), CAS#:2183512-02-7 (BODIPY® 630/650), CAS No.: 1818267-45-6 (BODIPY® 650/665), Difluoro(3-{2-[(5-phenyl-1H-pyrrol-2-yl-kappaN)methylene]-2H-pyrrol-5-yl-kappaN}propanoato)boron (BODIPY® R6G), CAS#:485397-12-4 (BODIPY® TMR), CAS#:2183473-18-7 (BODIPY® TR), SPK dye, cf514, DY405, DY396XL, cf570, cf405, conjugates thereof, and combinations thereof.

A dye is optionally a polymer dye. Illustrative examples of polymer dyes that may be used in any aspect provided herein include but are not limited to those described in U.S. Pat. No. 8,354,239, International application publication numbers WO2004/000970 or WO 2020/187919, or dyes sold as Brilliant Violet™ dyes by BD Biosciences (Franklin Lakes, NJ). In some aspects, the dye is Brilliant Violet 421™, Brilliant Violet 570™, Brilliant Violet 605™, Brilliant Violet 650™, Brilliant Violet 711™, or Brilliant Violet 785™.

Illustrative examples of dyes that may be used in any aspect provided herein include but are not limited to Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718.

A system as provided herein includes a leukocyte that is also incubated with a pro-aerolysin, or a derivative thereof. An aerolysin is a bacterial toxin that selectively binds GPI-anchors. Aerolysin is a channel-forming agent that causes cell lysis. A pro-aerolysin is a protoxin form of aerolysin that may be converted to an aerolysin by proteolytic cleavage by furin following binding to a GPI-anchor. (Rossjohn et al., J. Struct. Biol., 1998; 121:92-100.) In some aspects, as provided herein, a pro-aerolysin is mutated to form a derivative of pro-aerolysin such that the GPI-anchor binding properties are substantially present, but the toxin is either unable to be cleaved to form aerolysin or is otherwise deficient in channel forming such that binding to a leukocyte with GPI-anchors does not lead to death or lysis of the leukocyte by the action of the pro-aerolysin. Such pro-aerolysins are well known in the art as illustrated by Nakamura, et al., PLOS One, 2012, doi.org/10.1371/journal. pone.0033563. Optionally, a pro-aerolysin is variant (T253/A300C), which renders the cleaved aerolysin unable to form pores thereby inactivating the toxin (Brodsky, et al., Am J Clin Pathol, 2000; 114(3): 459-466). It is appreciated that other mutations in the loop or beta-sheet region of pro-aerolysin may also be used (See e.g. Rossjohn, et al., Biochemistry, 1998; 37:741-746). Alternatively, a pro-aerolysin derivative is mutated at the furin cleavage site of R432 or within 1-2 amino acids from R432. (See e.g. Abrami, et al., Journal of Biological Chemistry, 1998; 273(49):32656-32661.) It is known which locations on pro-aerolysin may be mutated to maintain GPI-anchor binding and eliminate or reduce toxin activity (e.g. pore forming) such as the mutations described by MacKenzie, et al., Journal of Biological Chemistry, 1999; 274(32):22604-22609. Illustratively, a pro-aerolysin may be as sold as FLAER by BioLegend, Inc. Illustratively, a pro-aerolysin is sold as Spark Blue 488 FLAER by Biolegend, Inc. (San Diego, CA). It is appreciated that any dye as provided herein may optionally be bound to a pro-aerolysin in place of or in addition to Sparke Blue 488.

As used herein, a blocking agent or a pro-aerolysin is optionally incubated with a leukocyte under conditions in which all or substantially all of the target GPI-anchored proteins or GPI-anchors are bound to either a blocking agent or a pro-aerolysin. As such, the concentration of the leukocyte, blocking agent, and pro-aerolysin may be chosen such that the incubation of the leukocyte with the blocking agent and the pro-aerolysin binds all or substantially all of the target of the blocking agent or pro-aerolysin.

A system as provided herein may include an aqueous component housing the leukocyte, blocking agent, dye-labeled antibody, or other. An aqueous component optionally includes a buffering composition. A buffering composition may include at least one buffering agent that stabilizes the pH of the system. Choice of buffering agent can be readily handled by one of ordinary skill in the art but buffering agents that have a pKa in the range of 6-8 are desirable for studies of many biological systems. Illustrative examples of such buffering agents include but are not limited to sodium bicarbonate, sodium carbonate, potassium bicarbonate, potassium carbonate, potassium phosphate, sodium phosphate, ethylene diamine, imidazole, triethanolamine, TRIS (tris(hydroxymethyl)aminomethane), HEPES (4-(2-Hydroxyethyl)-1-piperazineethanesulfonic acid), or any combination thereof. Optionally, a buffering agent excludes HEPES. A buffering agent is optionally present in the composition at a concentration of 0.01 millimolar (mM) to 10 molar (M), or any value or range therebetween. Optionally, a buffering agent is a phosphate buffering agent, illustratively sodium and/or potassium phosphates or mixture thereof as understood in the art optionally at 10 to 100 mM. Optionally, a buffering agent is phosphate buffered saline as this is understood in the art.

It is appreciated that buffering composition optionally further includes one or more salts as are typically included in a buffer as understood in the art, optionally NaCl, KCl, MgCl₂, among others. A salt is optionally present at a concentration of 0.1 mM to 250 mM, or any value or range therebetween.

A buffering composition may include one or more additives. An additive is optionally a protein additive. Illustrative examples of a protein additive include but are not limited to an albumin or the like. A protein additive is optionally an albumin, optionally bovine serum albumin or human serum albumin. A protein additive is optionally included at about 0.1 wt % to about 5 wt % or other desirable concentration. In some aspects, a protein additive is present at about 0.1 wt %, optionally about 0.25 wt %, optionally about 0.5 wt %, optionally about 0.75 wt %, optionally about 1.0 wt %, optionally about 1.25 wt %, optionally about 1.5 wt %, optionally about 1.75 wt %, optionally about 2.0 wt %, optionally about 2.25 wt %, optionally about 2.5 wt %, optionally about 2.75 wt %, optionally about 3.0 wt %, optionally about 3.25 wt %, optionally about 3.5 wt %, optionally about 3.75 wt %, optionally about 4.0 wt %, optionally about 4.25 wt %, optionally about 4.5 wt %, optionally about 4.75 wt %, optionally about 5.0 wt %.

The remainder of the buffering composition optionally includes water, optionally purified water or any water suitable for use in biological analyses such as flow cytometry and the like. Water is optionally any laboratory grade water (e.g. ASTM grades Type I, II, and III), reverse-osmosis water (RO), or deionized water (DI). Optionally, water is Type I water. Water is optionally RNAse free water meaning that the level of RNAse in the water is below detection limits. Optionally water is RNAse free Type I water.

In particular aspects, a system as provided herein may include a leukocyte including GPI-anchors, one or more antibody blocking agents or a fragment thereof each independently specific to one or more GPI-anchored proteins, and a pro-aerolysin or derivative thereof. An antibody may be any antibody as provided herein optionally defined herein as a blocking agent and optionally bound or not bound to one or more dyes optionally as presented herein. A blocking agent (e.g. antibody) may be specific for a GPI-anchored protein selected from the group of CD157, CD64, CD15, CD45, CD24, or CD14. Optionally, a blocking agent (e.g. antibody) may be specific for a GPI-anchored protein selected from the group of CD157, CD24, or CD14. In some aspects, the system includes a blocking agent (e.g. antibody) specific to CD157, a blocking agent (e.g. antibody) specific to CD24, and a blocking agent (e.g. antibody) specific to CD14.

Also provided are processes of detecting the presence or absence of paroxysmal nocturnal hemoglobinuria (PNH) in a subject that may include: obtaining a first sample of whole blood (or fraction thereof) from a subject, wherein the sample is optionally lysed, fixed, and/or washed; contacting the sample with a blocking solution including in one or more antibodies each independently specific to one or more GPI-anchored proteins and a pro-aerolysin or derivative thereof, and incubating for a first incubation time to form a blocked sample; combining the blocked sample with a PNH antibody cocktail; and subsequently subjecting the blocked sample to analysis by flow cytometry to detect the presence or absence of paroxysmal nocturnal hemoglobinuria in the subject.

It is appreciated that a sample may be whole blood or a fraction thereof. A fraction of whole blood may be plasma or serum. The preparation of plasma from whole blood is known in the art and may be achieved by centrifugation. The preparation of serum from either plasma or whole blood may be achieved as is known in the art by coagulating the whole blood or plasma and isolating the serum therefrom such as by centrifugation.

A sample may be lysed. Lysing of a sample of whole blood (or other such sample) is performed to reduce red blood cell contamination and may be achieved by methods as are known in the art illustratively as described by Sutherland, et al., Cytometry Part B (Clinical Cytometry) 2012; 82B:195-208. A lysing agent may be any known lysing agent, but optionally Optilyse C (Beckman Coulter), FACSLyse (BD Biosciences), or ammonium chloride based lysing agents.

A sample is optionally fixed by techniques as illustratively known in the art. Optionally, a sample is fixed using a lysing agent that includes one or more known fixatives to help promote cellular integrity for subsequent incubation with one or more blocking agents and or pro-aerolysin as otherwise provided herein.

Following lysing and/or fixing, a sample is optionally washed such in a wash buffer that is or includes a buffering composition as otherwise disclosed herein. A wash buffer optionally is or includes phosphate buffered saline (PBS).

A sample is contacted with a blocking solution that includes one or more blocking agents, optionally antibodies or fragments thereof as provided herein, wherein each antibody or fragment thereof is independently specific for one or more GPI-anchored proteins, and a pro-aerolysin each as otherwise provided herein. The sample is incubated with the one or more GPI-anchored proteins and a pro-aerolysin for an incubation time to form a blocked sample. It is appreciated that the concentration of the one or more antibodies (blocking agents) and pro-aerolysin is each independently sufficient to substantially block all or about all of the targets of the antibody or pro-aerolysin (e.g. GPI-anchored protein or GPI-anchor, respectively). Optionally, a blocking agent is present at a concentration of 0.1 microgram/microliter (μg/μl) to 1 μg/μl or any value or range therebetween, optionally 0.2 to 0.6 μg/μl. An incubation time is optionally 1 minute (min) to 30 min or more. Optionally, an incubation time is 1, 2, 3, 4, 5, 6, 7, 8, 9, 10, 11, 12, 13, 14, 15, 16, 17, 18, 19, 20, 21, 22, 23, 24, 25, 26, 27, 29, 29, or 30 min. Optionally, an incubation time is 10 min to 20 min, or any value or range therebetween. An incubation is optionally at an incubation temperature. An incubation temperature is optionally about 20° C. to about 38° C., or any value or range therebetween. Optionally, an incubation temperature is about 37.5° C. Optionally, an incubation temperature is about 25° C.

Following incubation with a blocking agent (antibody) and a pro-aerolysin for the incubation time to form a blocked sample, the blocked sample is combined with a PNH antibody cocktail. A PNH antibody cocktail may include one or more antibodies (or fragment thereof) specific to a GPI-anchored protein wherein the antibody is also bound to one or more dyes optionally as provided herein. A PNH antibody cocktail optionally includes one or more antibodies specific for a GPI-anchored protein. Optionally, a PNH cocktail includes an antibody or fragment thereof specific for CD157, CD64, CD15, CD45, CD24, and/or CD14. In some aspects, a PNH cocktail includes 1, 2, 3, 4, 5, 6, 7, 8, 9, or more antibodies. Optionally, a PNH cocktail includes six antibodies, optionally wherein each of the six antibodies are independently specific for CD157, CD64, CD15, CD45, CD24, or CD14. A PNH antibody cocktail may further include the antibodies or fragments thereof in a buffer composition as provided herein. The concentration of the antibodies in the PNH antibody cocktail are sufficient to substantially saturate all binding sites of each antibody on the cells of the sample sufficiently to detect the presence or absence of binding of the antibodies to the PNH antibody cocktail to the leukocyte within the sample.

In some aspects, the PNH antibody cocktail used in a method of analyzing a biological sample includes two or more antibodies specific for different GPI-anchored proteins or for any other cell surface protein that may be used alone or in combination with other antibodies useful to identity of the cell being studied. Optionally, an antibody specific for a GPI-anchored protein is conjugated to a first dye, and wherein an antibody specific for a second GPI-anchored protein is conjugated to a second dye, where the first dye and the second dye may be characterized by different fluorescent properties. Optionally, each antibody in a PNH cocktail is bound to a different dye.

The process includes subjecting the blocked sample that has been incubated with the PNH cocktail to analysis by an analytical method to detect the presence or absence of paroxysmal nocturnal hemoglobinuria (or other disease or condition) in the subject. The analytical method may utilize one or more various analytical methods illustratively but not limited to flow cytometry, FISH, immunohistochemistry, sandwich assay, Southern blot, western blot, microarray, or substrate binding assay. In some aspects, an analytical method is flow cytometry.

In some aspects, a process of detecting the presence or absence of paroxysmal nocturnal hemoglobinuria (PNH) in a subject further includes obtaining a second sample of whole blood or a fraction thereof, combining the second sample with a second PNH antibody cocktail (optionally of the same composition as a first PNH antibody cocktail), and intermixing the second sample with the blocked sample prior to subjecting the intermixed sample to analysis by flow cytometry. The second sample is optionally a portion of the first sample. The second sample is optionally not incubated or otherwise contacted with an antibody specific to a GPI-anchored protein or a pro-aerolysin or derivative thereof. In specific aspects, a single sample is obtained from a subject and split into two or more separate subsamples, wherein the first subsample is incubated with the antibody specific to one or more GPI-anchored proteins and a pro-aerolysin or derivative thereof and the second subsample is not. Thus, the overall sample analysis includes some portions of the sample that are blocked by the incubation of the anti-GPI-anchored protein antibodies and the pro-aerolysin and some portion of the original sample not blocked by the incubation of the anti-GPI-anchored protein antibodies and the pro-aerolysin. In such a system, the amount of cells of the original sample from the subject not bound to the anti-GPI-anchored protein antibodies and the pro-aerolysin relative to the cells that are bound to the anti-GPI-anchored protein antibodies and the pro-aerolysin may optionally effectively mimic the ratio of such cells in a subject that is positive for PNH and wherein the sample may act as a positive control in the system to improve detectability and accuracy of the analyses.

As such, in some aspects also provided are processes of analyzing a positive control for PNH including: obtaining a leukocyte including GPI-anchors, the leukocyte incubated with one or more antibodies (or other blocking agent) each independently specific to one or more GPI-anchored proteins, further incubating the leukocyte with a pro-aerolysin or derivative thereof to form a blocked leukocyte; further incubating the blocked leukocyte with a PNH antibody cocktail; combining the blocked leukocyte with an unblocked leukocyte to form a leukocyte mix; and subjecting the leukocyte mix to analysis by one or more analytical methods as provided herein, optionally flow cytometry. A leukocyte optionally is or includes one or more monocytes. Optionally, a leukocyte is or includes one or more granulocytes. Optionally, a leukocyte is a plurality of leukocytes that may be monocytes, granulocytes, or a combination thereof. In some aspects, a plurality of leukocytes are or include monocytes. The monocytes, granulocytes or combination thereof are optionally human monocytes, granulocytes or a combination thereof. In some aspects, a leukocyte is a previously lyophilized leukocyte. The leukocytes may be lyophilized or lyophilized previously and reconstituted in a reconstitution buffer. Illustrative examples of lyophilized leukocytes are those sold as VERI CELL by Biolegend, Inc. (San Diego, CA). As such, a process of analyzing a positive control for paroxysmal nocturnal hemoglobinuria (PNH) using the blocked leukocytes as provided herein allows for a sample to resemble the signal obtained when a sample from a subject with PNH is analyzed by the same system and using the same detection agents (e.g. antibodies) and thereby acts as a positive control for PNH in the instrumentation of the analytical method and improving confidence in the result achieved using a sample from a subject with unknown PNH status.

Also provided are kits. A kit may optionally be used in a process of detecting the presence or absence of PNH in a subject and/or optionally in forming a positive control. A kit as provided herein optionally includes a first container and a second container, wherein the first container includes an unblocked leukocyte that is or is configured to be incubated with a PNH antibody cocktail; and wherein the second container includes a blocked leukocyte, the blocked leukocyte incubated or configured to be incubated with one or more antibodies to one or more GPI-anchored proteins, a pro-aerolysin or derivative thereof, and with the PNH antibody cocktail. The amount of cells of the unblocked leukocyte not bound to the anti-GPI-anchored protein antibodies and the pro-aerolysin relative to the blocked leukocytes may effectively mimic the ratio of such cells in a subject that is positive for PNH. Such kits can readily, rapidly and reproducibly be used in processes of detection of the presence or absence of paroxysmal nocturnal hemoglobinuria (PNH) in a subject.

On other aspects, a kit includes: a first container containing leukocytes, optionally lyophilized leukocytes; a second container containing leukocytes, optionally lyophilized leukocytes; a blocking agent solution; a reconstitution buffer; and a PNH antibody cocktail as provided herein, optionally further including a labeled pro-aerolysin. A reconstitution buffer is optionally water or any buffer suitable for reconstitution lymphocytes, optionally a PBS buffer optionally further including a polymer such as PVP. A reconstitution buffer is optionally as described in U.S. Pat. No. 5,641,637. Optionally a reconstitution buffer is Buffer A Plus as sold by Biolegend, Inc. (San Diego, CA). A blocking agent solution includes one or more blocking agents as described herein in a reconstitution buffer or other suitable buffer.

Various aspects of the present disclosure are illustrated by the following non-limiting examples. The examples are for illustrative purposes and are not a limitation on any practice of the present disclosure. It will be understood that variations and modifications can be made without departing from the spirit and scope of the disclosure. Reagents illustrated herein are commercially available or readily synthesized by well-known methods from readily commercially available precursors, and a person of ordinary skill in the art readily understands where such reagents may be obtained.

### EXAMPLES

### Example 1

Samples of whole blood were obtained by venipuncture from consenting adults that were not diagnosed with PNH. Samples were collected into vacutainer blood collection tubes containing heparin as the anticoagulant (BD Biosciences, Franklin Lakes, NJ, USA). Samples were immediately analyzed.

Spark Blue™ 488 FLAER (pro-aerolysin conjugated to AlexaFlour® 488) was obtained from BioLegend, Inc. (San Diego, CA). Phycoerythrin (PE)-conjugated anti-CD157 (clone W21007F), Brilliant Violet 421 conjugated CD45 (clone 2D1), PE-cyanine (Cy)7-conjugated anti-CD64 (clone S18012C), PerCp Cy5.5 conjugated anti-CD15 (clone HI98), allophycocyanin (APC)-conjugated anti-CD24 (clone ML5) and APC Cy7 conjugated anti-CD14 (clone S18004B) were all produced in house at Biolegend, Inc. (San Diego, CA).

100 μl Heparin-whole blood samples were each transferred to Falcon® 5 mL Round Bottom Polystyrene Test Tubes (#35205) and subjected to lysis using 2 mL 1× RBC Lysis/Fixation Solution (BioLegend Cat. No. 422401) followed by washing in PBS and resuspending in 100 μl Cell Staining Buffer. In a first tube, the cells were blocked with a combination of anti-CD157(15 , 30, or 40 micrograms (μg)), anti-CD14 (15 μg), APC-conjugated anti-CD24(10, 15, 30, or 40 μg), and pro-aerolysin (15 μg) in PBS. Blocking was performed at about 25° C. for 15 minutes. The cells were then stained with a PNH antibody cocktail that included PE-conjugated anti-CD157 (1 μg), APC Cy7 conjugated anti-CD14 (0.5 μg), APC-conjugated anti-CD24 (0.5 μg), Brilliant Violet 421 conjugated CD45 (0.25 μg), PE-cyanine (Cy)7-conjugated anti-CD64(0.25 μg) and PerCp Cy5.5 conjugated anti-CD15(0.5 μg) in PBS incubated at about 25° C. for 15 minutes, and washed in PBS.

A second tube of fixed/lysed sample was combined with PBS and incubated at about 25° C. for 15 minutes followed by addition of the above PNH antibody cocktail and incubated at about 25° C. for 15 minutes, then washed in PBS. The cells of the first tube and the cells of the second tube were then intermixed at a cell number ratio of about 1:1.

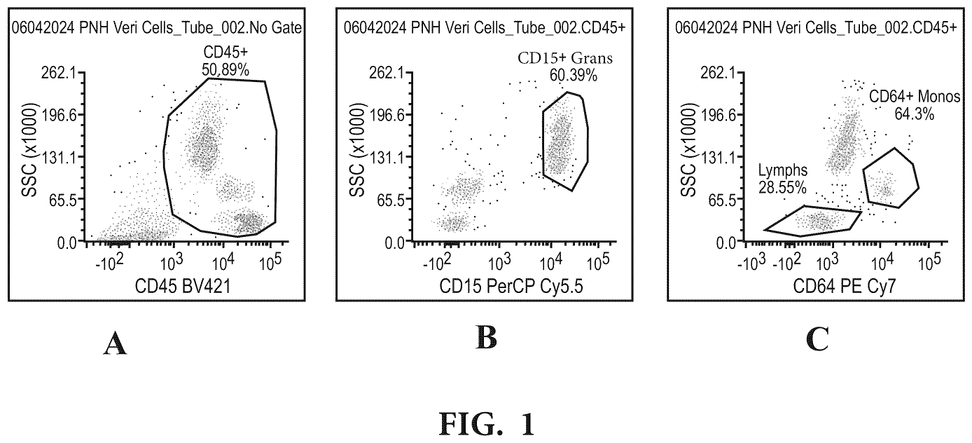

The intermixed cells were applied to a BD FACS CANTO flow cytometer and counted until a total of 10,000 cells were detected. Monocyte counts were detected by positive CD64 binding. Granulocytes were detected by positive CD15 binding. Total lymphocytes were detected by staining with FLAER. As illustrated in FIG. 1, monocytes and granulocytes were gated together on a CD45/SSC plot.

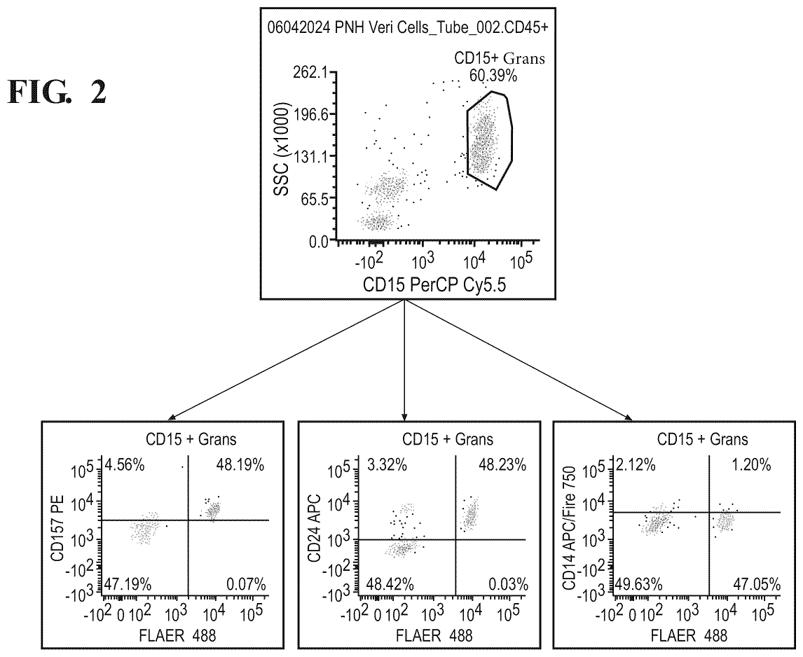

Granulocytes were identified by positive anti-CD15 binding (B) and monocytes were identified by positive anti-CD64 binding. The population of granulocytes of FIG. 1(B) were analyzed for anti-CD157, anti-CD24, or anti-CD14 binding each individually against FLAER 488. Two separate populations of cells were identified in each plot demonstrating effective blocking of each of CD14, CD157, and CD24 as well as pro-aerolysin as illustrated in FIG. 2.

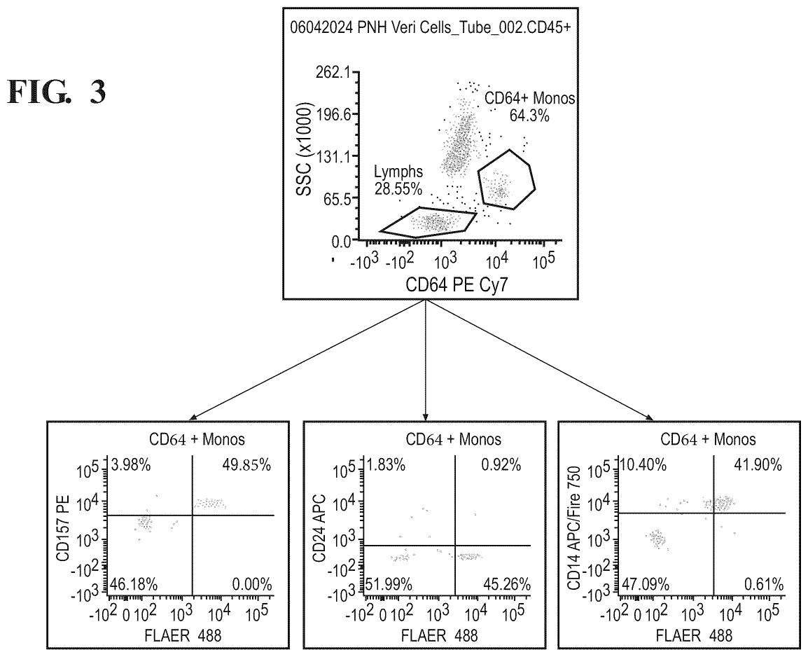

Monocytes were identified by positive anti-CD64 binding FIG. 1(C). The population of monocytes of FIG. 1(C) were analyzed for anti-CD157, anti-CD24, or anti-CD14 binding each individually against FLAER 488. Two separate populations of cells, one double positive and one double negative, were identified in each plot demonstrating effective blocking of each of CD14, CD157, and CD24 as well as pro-aerolysin as illustrated in FIG. 3.

### Example 2

Additional studies were performed using the methods of Example 1 to determine if previously lyophilized leukocytes (Veri Cells, Biolegend, Inc., San Diego, CA) could be effectively blocked so as to be used as a positive control for PNH detection.

A first container of Veri Cells was reconstituted in Buffer A+(or PBS) to a concentration of (≥5×10⁶ cells/50 μl). These reconstituted Veri Cells were then incubated with the PHC antibody cocktail as in Example 1 at about 25° C. for 15 minutes followed by washing and resuspending in PBS.

A second container of Veri Cells were reconstituted in Buffer C (includes anti-CD157 (15, 30, or 40 micrograms (μg)), anti-CD14 (15 μg), APC-conjugated anti-CD24(10, 15, 30, or 40 μg), and pro-aerolysin (15 μg) all in Buffer A+) (≥5×10⁶ cells/50 μl). Blocking was performed at about 25° C. for 15 minutes. These blocked Veri Cells were then incubated with the PNH antibody cocktail as in Example 1 at about 25° C. for 15 minutes followed by washing and resuspending in PBS.

The first container and the second container were then intermixed at a cell number ratio of about 1:1 and analyzed by flow cytometry.

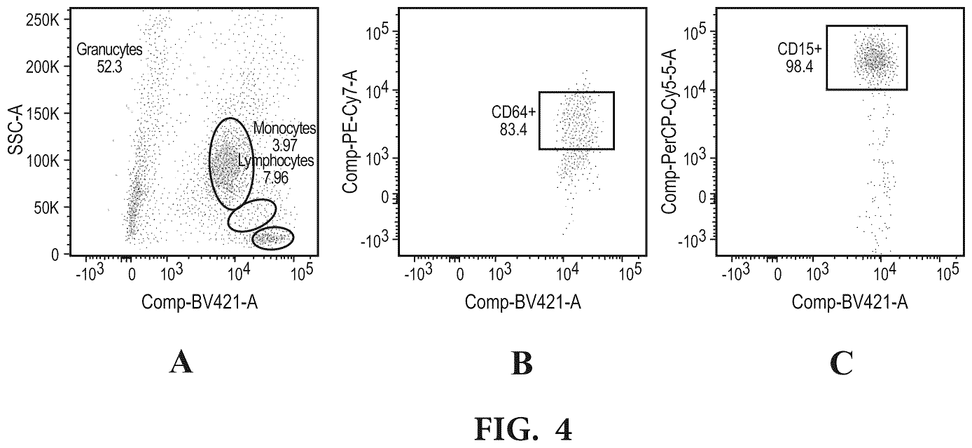

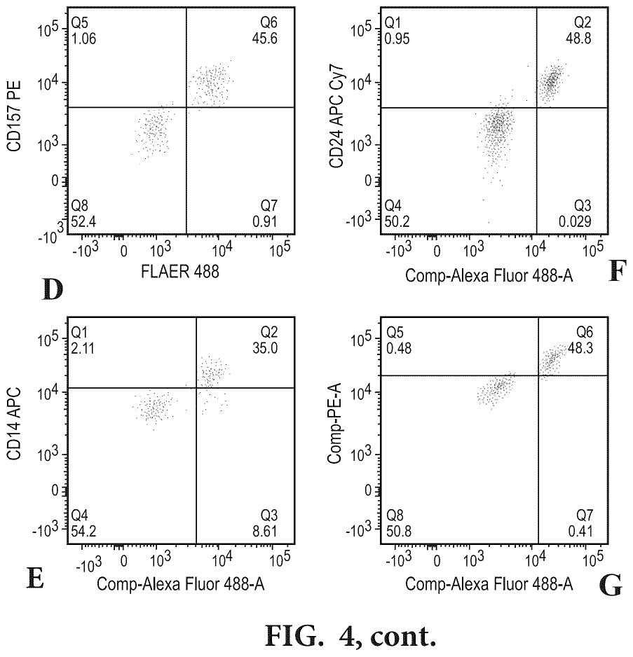

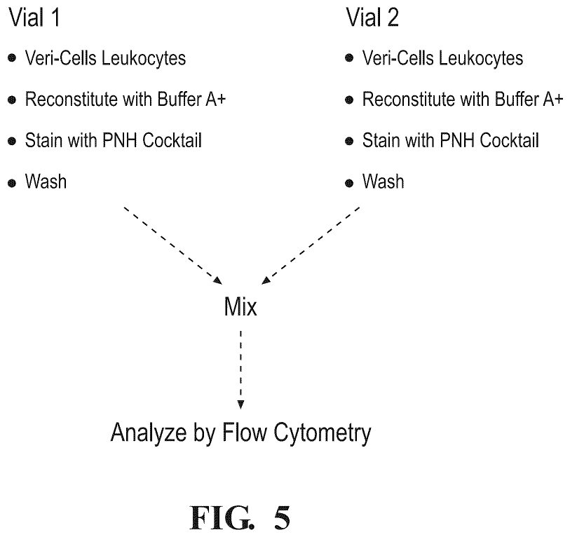

As illustrated in FIG. 4, Veri Cells were successfully able to be blocked and mimic a PNH sample. FIG. 4(A) illustrates monocytes and granulocytes identified on a CD45/SSC plot. Granulocytes were identified by positive anti-CD15 binding (C) and monocytes were identified by positive anti-CD64 binding (B). The population of granulocytes of FIG. 4(C) were analyzed for anti-CD157 (F) or anti-CD24 (G) binding each individually against FLAER 488. Two separate populations of cells were identified in each plot demonstrating effective blocking of each of CD157 and CD24 as well as pro-aerolysin as illustrated in FIG. 4.

Monocytes were identified by positive anti-CD64 binding FIG. 4(B). The population of monocytes of FIG. 4(B) were analyzed for anti-CD157 (D) and anti-CD14 (E) binding against FLAER 488. Two separate populations of cells were identified in each plot demonstrating effective blocking of each of CD14 and CD157 as well as pro-aerolysin as illustrated in FIG. 4.

### Additional Exemplary Aspects

Exemplary Aspect 1. A system for use in detecting the presence or absence of paroxysmal nocturnal hemoglobinuria (PNH) in a subject, said detecting comprising:
obtaining a first sample of whole blood from said subject, said sample optionally red blood cell lysed, fixed, and washed, said first sample comprising one or more leukocytes;
contacting said first sample with a blocking solution comprising
   one or more blocking agents each independently specific to one or more GPI-anchored proteins, and
   a pro-aerolysin or derivative thereof;
incubating said sample with said blocking solution for a first incubation time to form a blocked sample;
combining said blocked sample with a PNH antibody cocktail; and
subsequently subjecting said blocked sample to analysis by flow cytometry to detect the presence or absence of paroxysmal nocturnal hemoglobinuria in said subject.

Exemplary Aspect 2. The system of Exemplary Aspect 1 further comprising:
obtaining a second sample of whole blood from said subject, said second sample optionally a portion of said first sample;
combining said second sample with the PNH antibody cocktail; and
intermixing said second sample with said blocked sample prior to subjecting said blocked sample to analysis by flow cytometry.

Exemplary Aspect 3. The system of Exemplary Aspect 2, wherein said second sample is a portion of said first sample.

Exemplary Aspect 4. The system of Exemplary Aspects 1 or 2, wherein one or more blocking agents to one or more GPI-anchored proteins is specific for binding to CD157, CD24, or CD14.

Exemplary Aspect 5. The system of Exemplary Aspect 4, wherein said one or more blocking agents to one or more GPI-anchored proteins comprises an antibody specific to CD157, an antibody specific to CD24, and an antibody specific to CD14.

Exemplary Aspect 6. The system of any one of Exemplary Aspects 1-5, wherein said PNH antibody cocktail comprises a labeled antibody specific for CD157, CD64, CD15, CD45, CD24, or CD14.

Exemplary Aspect 7. The system of Exemplary Aspect 6, wherein said PNH antibody cocktail comprises six antibodies, wherein each of said six antibodies are independently specific for CD157, CD64, CD15, CD45, CD24, and CD14.

Exemplary Aspect 8. The system of any one of Exemplary Aspects 1-7 comprising:
one or more blocking agents each independently specific to one or more GPI-anchored proteins, optionally wherein said blocking agents are incubated with a first leukocyte from said sample; and
a pro-aerolysin or derivative thereof.

Exemplary Aspect 9. The system of Exemplary Aspect 8, wherein one or more of said blocking agents is specific for binding to CD157, CD64, CD15, CD45, CD24, or CD14.

Exemplary Aspect 10.The system of Exemplary Aspect 8, wherein one or more of said blocking agents is specific for binding to CD157, CD24, or CD14.

Exemplary Aspect 11.The system of claim 8, wherein said system comprises said leukocytes incubated with a blocking agent specific for binding to CD157, a blocking agent specific for binding to CD24, and a blocking agent specific for binding to CD14.

Exemplary Aspect 12.The system of any one of Exemplary Aspects 8-11, wherein said leukocytes comprise a plurality of monocytes, granulocytes, or a combination thereof.

Exemplary Aspect 13.The system of Exemplary Aspect 12, wherein said leukocytes comprise monocytes.

Exemplary Aspect 14.The system of any one of Exemplary Aspects 8-13, wherein said leukocytes are human leukocytes.

Exemplary Aspect 15.The system of any one of Exemplary Aspects 8-13, wherein said leukocytes comprises previously lyophilized leukocytes.

Exemplary Aspect 16.The system of any one of Exemplary Aspects 8-13, wherein
said one or more blocking agents comprise one or more antibodies, said one or more antibodies each independently specific to one or more GPI-anchored proteins; and
said first leukocyte further incubated with said pro-aerolysin or derivative thereof.

Exemplary Aspect 17.The system of Exemplary Aspect 16, wherein one or more of said antibodies is specific for CD157, CD64, CD15, CD45, CD24, or CD14.

Exemplary Aspect 18.The system of Exemplary Aspect 16, wherein one or more antibodies is specific for CD157, CD24, or CD14.

Exemplary Aspect 19.The system of Exemplary Aspect 16, wherein said system comprises an antibody specific to CD157, and antibody specific to CD24, and an antibody specific to CD14.

Exemplary Aspect 20.The system of any one of Exemplary Aspects 16-19, wherein said leukocytes comprise a plurality of monocytes granulocytes, or a combination thereof.

Exemplary Aspect 21. The system of any one of Exemplary Aspects 16-20, wherein said leukocytes comprise monocytes.

Exemplary Aspect 22. The system of any one of Exemplary Aspects 16-20, wherein said leukocytes are human leukocytes.

Exemplary Aspect 23.The system of any one of Exemplary Aspects 16-20, wherein said leukocytes comprises previously lyophilized leukocytes.

Exemplary Aspect 24.The system of any one of Exemplary Aspects 16-20 further comprising one or more buffers, a lysing agent, or a combination thereof.

Exemplary Aspect 25.A process for analyzing a positive control for paroxysmal nocturnal hemoglobinuria (PNH) comprising:
obtaining a leukocyte comprising GPI-anchors, said leukocyte incubated with one or more blocking antibodies each independently specific to one or more GPI-anchored proteins;
further incubating said leukocyte with a pro-aerolysin or derivative thereof to form a blocked leukocyte;
further incubating said blocked leukocyte with a PNH antibody cocktail;
combining the blocked leukocyte with an unblocked leukocyte to form a leukocyte mix; and
subjecting said leukocyte mix to analysis by flow cytometry.

Exemplary Aspect 26.The process of Exemplary Aspect 25, wherein said leukocyte or said unblocked leukocyte comprises a plurality of monocytes, granulocytes, or a combination thereof.

Exemplary Aspect 27.The process of any one of Exemplary Aspects 25-26, wherein said blocked leukocytes and/or unblocked leukocytes are human leukocytes.

Exemplary Aspect 28.The process of any one of Exemplary Aspects 25-27, wherein said blocked leukocytes and/or unblocked leukocytes are previously lyophilized leukocytes.

Exemplary Aspect 29.The process of any one of Exemplary Aspects 25-28, wherein one or more of said blocking antibodies is specific for CD157, CD24, or CD14.

Exemplary Aspect 30.The process of Exemplary Aspect 29, wherein said blocking antibodies specific to one or more GPI-anchored proteins comprise three antibodies independently specific to CD157, CD24, and CD14.

Exemplary Aspect 31.The process of any one of Exemplary Aspects 25-30, wherein said PNH antibody cocktail comprises an antibody specific for CD157, CD64, CD15, CD45, CD24, or CD14.

Exemplary Aspect 32.The process of Exemplary Aspect 31, wherein said PNH antibody cocktail comprises six antibodies, wherein each of said six antibodies are independently specific for CD157, CD64, CD15, CD45, CD24, and CD14.

Exemplary Aspect 33.A kit for detection of the presence or absence of paroxysmal nocturnal hemoglobinuria (PNH) in a subject comprising:
a first container and a second container;
   said first container comprising an unblocked leukocyte said unblocked leukocyte optionally incubated with a PNH antibody cocktail; and
   said second container comprising a blocked leukocyte, said blocked leukocyte incubated with one or more blocking agent antibodies to one or more GPI-anchored proteins, a pro-aerolysin or derivative thereof, and with the PNH antibody cocktail.

Exemplary Aspect 34. A kit for detection of the presence or absence of paroxysmal nocturnal hemoglobinuria (PNH) in a subject comprising:
a first container containing leukocytes, optionally lyophilized leukocytes;
a second container containing leukocytes, optionally lyophilized leukocytes;
a blocking agent solution comprising one or more blocking agents;
a reconstitution buffer; and
a PNH antibody cocktail, optionally further including a labeled pro-aerolysin.

Exemplary Aspect 35.The kit of either of Exemplary Aspects 33 or 34, wherein said leukocytes comprise a plurality of monocytes, granulocytes, or a combination thereof.

Exemplary Aspect 36.The kit of any one of Exemplary Aspects 33-35, wherein said leukocytes are human leukocytes.

Exemplary Aspect 37.The kit of any one of Exemplary Aspects 33-36, wherein said leukocytes are previously lyophilized leukocytes.

Exemplary Aspect 38.The kit of any one of Exemplary Aspects 33-37, wherein one or more of said blocking agents is specific for CD157, CD24, or CD14.

Exemplary Aspect 39.The kit of Exemplary Aspect 38, wherein said blocking agent comprises three antibodies independently specific to CD157, CD24, and CD14.

Exemplary Aspect 40.The kit of any one of Exemplary Aspects 33-39, wherein said PNH antibody cocktail comprises an antibody specific for CD157, CD64, CD15, CD45, CD24, or CD14.

Exemplary Aspect 41.The kit of any one of Exemplary Aspects 33-39, wherein said PNH antibody cocktail comprises six antibodies, wherein each of said six antibodies are independently specific for CD157, CD64, CD15, CD45, CD24, and CD14.

Exemplary Aspect 42.A system configured for use in detection of the presence or absence of paroxysmal nocturnal hemoglobinuria comprising:
a leukocyte comprising GPI-anchors said leukocyte incubated with one or more blocking agents each independently specific to one or more GPI-anchored proteins;
said leukocyte further incubated with a pro-aerolysin or derivative thereof; and
optionally a second leukocyte comprising GPI-anchors not incubated with one or more of said blocking agents.

Exemplary Aspect 43.The system of Exemplary Aspect 42, wherein one or more of said blocking agents is specific for CD157, CD64, CD15, CD45, CD24, or CD14.

Exemplary Aspect 44.The system of Exemplary Aspect 42, wherein one or more of said blocking agents is specific for CD157, CD24, or CD14.

Exemplary Aspect 45.The system of Exemplary Aspect 42, wherein said system comprises said leukocytes incubated with a blocking agent specific to CD157, a blocking agent specific to CD24, and a blocking agent specific to CD14.

Exemplary Aspect 46.The system of any one of Exemplary Aspects 42-45, wherein said leukocytes comprise a plurality of monocytes, granulocytes, or a combination thereof.

Exemplary Aspect 47.The system of any one of Exemplary Aspects 42-46, wherein said leukocytes comprise monocytes.

Exemplary Aspect 48.The system of any one of Exemplary Aspects 42-47, wherein said leukocytes are human leukocytes.

Exemplary Aspect 49.The system of any one of Exemplary Aspects 42-48, wherein said leukocytes comprises previously lyophilized leukocytes.

Exemplary Aspect 50.A system configured for use in detection of the presence or absence of paroxysmal nocturnal hemoglobinuria comprising:
a leukocyte comprising GPI-anchors;
one or more antibodies each independently specific to one or more GPI-anchored proteins; and
a pro-aerolysin or derivative thereof.

Exemplary Aspect 51.The system of Exemplary Aspect 50, wherein one or more of said antibodies is specific for CD157, CD64, CD15, CD45, CD24, or CD14.

Exemplary Aspect 52.The system of Exemplary Aspect 50, wherein one or more antibodies is specific for CD157, CD24, or CD14.

Exemplary Aspect 53.The system of Exemplary Aspect 50, wherein said system comprises an antibody specific to CD157, an antibody specific to CD24, and an antibody specific to CD14.

Exemplary Aspect 54.The system of any one of Exemplary Aspects 50-53, wherein said leukocytes comprise a plurality of monocytes granulocytes, or a combination thereof.

Exemplary Aspect 55.The system of any one of Exemplary Aspects 50-54, wherein said leukocytes comprise monocytes.

Exemplary Aspect 56.The system of any one of Exemplary Aspects 50-55, wherein said leukocytes are human leukocytes.

Exemplary Aspect 57.The system of any one of Exemplary Aspects 50-56, wherein said leukocytes comprises previously lyophilized leukocytes.

Exemplary Aspect 58.The system of any one of Exemplary Aspects 50-57 further comprising one or more buffers, a lysing agent, or a combination thereof.

Exemplary Aspect 59.A process of detecting the presence or absence of a disease or condition substantially as described herein.

Exemplary Aspect 60.A system for detecting the presence or absence of a disease or condition comprising one or more blocking agents substantially as described herein.

The foregoing description of particular aspect(s) is merely exemplary in nature and is in no way intended to limit the scope of the disclosure, or any related invention, its application, or uses, which may, of course, vary. The compositions or processes are described with relation to the non-limiting definitions and terminology included herein. These definitions and terminology are not designed to function as a limitation on the scope or practice of any invention disclosed herein but are presented for illustrative and descriptive purposes only. While the processes or compositions are described as an order of individual steps or using specific materials, it is appreciated that steps or materials may be interchangeable such that the description may include multiple parts or steps arranged in many ways as is readily appreciated by one of skill in the art.

Definitions as used herein are intended to supplement and illustrate, not preclude, the definitions known to those of skill in the art.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Any patents or publications mentioned in this specification are indicative of the levels of those skilled in the pertinent art. These patents and publications are herein incorporated by reference to the same extent as if each individual publication was specifically and individually indicated to be completely incorporated by reference.

One skilled in the art will readily appreciate that the present disclosure is well-adapted to carry out the objects and obtain the ends and advantages mentioned, as well as those inherent therein. The present examples along with the methods, procedures, treatments, molecules and specific compounds described herein are presently representative of specific aspects, are exemplary, and are not intended as limitations on the scope of any invention, which shall be solely defined by the foregoing claims. It will be apparent that other aspects exist and are encompassed within the spirit of any invention as defined by the scope of the claims.

We claim:

1. A system configured for use in detection of the presence or absence of paroxysmal nocturnal hemoglobinuria comprising:
a first leukocyte comprising GPI-anchors, said first leukocyte incubated with one or more blocking agents each independently specific to one or more GPI-anchored proteins; and
said leukocyte further incubated with a pro-aerolysin or derivative thereof.

2. The system of claim 1, wherein one or more of said blocking agent is specific for CD157, CD64, CD15, CD45, CD24, or CD14.

3. The system of claim 1, wherein said leukocytes comprise a plurality of monocytes, granulocytes, or a combination thereof.

4. The system of claim 1, wherein said leukocytes comprise previously lyophilized leukocytes.

5. The system of claim 1, further comprising a second leukocyte comprising GPI-anchors not incubated with one or more of said blocking agents.

6. The system of claim 1 further comprising one or more buffers, a lysing agent, or a combination thereof.

7. A process of detecting the presence or absence of paroxysmal nocturnal hemoglobinuria (PNH) in a subject comprising:
obtaining a first sample of whole blood from said subject, said sample optionally lysed, fixed, and washed;
contacting said sample with a blocking solution comprising one or more blocking antibodies each independently specific to one or more GPI-anchored proteins and a pro-aerolysin or derivative thereof, and incubating for a first incubation time to form a blocked sample;
combining said blocked sample with a PNH antibody cocktail; and
subsequently subjecting said blocked sample to analysis by flow cytometry to detect the presence or absence of paroxysmal nocturnal hemoglobinuria in said subject.

8. The process of claim 7 further comprising:
obtaining a second sample of whole blood from said subject;
combining said second sample with the PNH antibody cocktail; and
intermixing said second sample with said blocked sample prior to subjecting said blocked sample to analysis by flow cytometry.

9. The process of claim 8, wherein said second sample is a portion of said first sample.

10. The process of 7, wherein the one or more blocking antibodies to one or more GPI-anchored proteins is specific for CD157, CD24, or CD14.

11. The process of claim 10, wherein said one or more blocking antibodies to one or more GPI-anchored proteins comprises an antibody specific to CD157, an antibody specific to CD24, and an antibody specific to CD14.

12. The process of claim 7, wherein said PNH antibody cocktail comprises an labeled antibody specific for CD157, CD64, CD15, CD45, CD24, or CD14.

13. The process of claim 12, wherein said PNH antibody cocktail comprises six antibodies, wherein each of said six antibodies are independently specific for CD157, CD64, CD15, CD45, CD24, and CD14.

14. A process of analyzing a positive control for paroxysmal nocturnal hemoglobinuria (PNH) comprising:
obtaining a leukocyte comprising GPI-anchors, said leukocyte incubated with one or more blocking antibodies each independently specific to one or more GPI-anchored proteins;
further incubating said leukocyte with a pro-aerolysin or derivative thereof to form a blocked leukocyte;
further incubating said blocked leukocyte with a PNH antibody cocktail;
combining the blocked leukocyte with an unblocked leukocyte to form a leukocyte mix; and
subjecting said leukocyte mix to analysis by flow cytometry.

15. The process of claim 14, wherein said leukocyte or said unblocked leukocyte comprise a plurality of monocytes, granulocytes, or a combination thereof.

16. The process of claim 14, wherein said blocked leukocytes and/or unblocked leukocytes are previously lyophilized leukocytes.

17. The process of claim 14, wherein one or more of said blocking antibodies is specific for CD157, CD24, or CD14.

18. The process of claim 17, wherein said blocking antibodies specific to one or more GPI-anchored proteins comprises three antibodies independently specific to CD157, CD24, and CD14.

19. The process of claim 14, wherein said PNH antibody cocktail comprises an antibody specific for CD157, CD64, CD15, CD45, CD24, or CD14.

20. The process of claim 19, wherein said PNH antibody cocktail comprises six antibodies, wherein each of said six antibodies are independently specific for CD157, CD64, CD15, CD45, CD24, and CD14.
